(19) United States
(12) Patent Application Publication
(10) Pub. No.: US 2024/0112804 A1
(43) Pub. Date: Apr. 4, 2024

(54) MATCHING UNSTRUCTURED TEXT TO CLINICAL ONTOLOGIES

(71) Applicant: Google LLC, Mountain View, CA (US)

(72) Inventors: Itay Laish, Timrat (IL); Uri N. Lerner, Los Altos, CA (US); Aviel Atias, Tel Aviv (IL); Natan Potikha, Tel Aviv (IL); Ayelet Benjamini, Kfar Saba (IL)

(21) Appl. No.: 18/375,914

(22) Filed: Oct. 2, 2023

Related U.S. Application Data
(60) Provisional application No. 63/412,342, filed on Sep. 30, 2022.

(51) Int. Cl. G16H 50/20; G06F 40/166; G06F 40/295; G16H 10/60; G16H 15/00

(52) U.S. Cl. CPC G16H 50/20; G06F 40/166; G06F 40/295; G16H 10/60; G16H 15/00

(57) ABSTRACT

A computer-implemented method for matching unstructured text to ontology entities in a clinical ontology is described. The method includes receiving one or more clinical notes associated with a patient; for each of the one or more clinical notes: extracting, using a neural network, one or more text spans from unstructured text in each clinical note, each of the one or more text spans identifying a respective input phrase in the unstructured text; for each of the one or more text spans, matching, using a text matcher, the text span with a respective output ontology entity from an ontology, the respective output ontology entity relating to a clinical condition of the patient; and outputting data defining the one or more text spans and the respective output ontology entity for each of the one or more text spans.

### CROSS REFERENCE TO RELATED APPLICATIONS

This application is a non-provisional of and claims priority to U.S. Provisional Patent Application No. 63/412,342, filed on Sep. 30, 2022, the entire contents of which are hereby incorporated by reference.

### BACKGROUND

This specification relates to neural networks for matching unstructured text to ontology entities in a clinical ontology.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system implemented as computer programs on one or more computers in one or more locations for matching unstructured text in clinical notes to ontology entities in an ontology in order to determine clinical conditions of a patient.

In general, one innovative aspect of the subject matter described in this specification can be embodied in a computer-implemented method for matching unstructured text in clinical notes to ontology entities in an ontology. The method includes receiving one or more clinical notes associated with a patient, wherein each of the one or more clinical notes includes unstructured text. The method includes, for each of the one or more clinical notes: extracting, using a neural network, one or more text spans from the unstructured text, each of the one or more text spans identifying a respective input phrase in the unstructured text; for each of the one or more text spans, matching, using a text matcher, the text span with a respective output ontology entity from an ontology, the respective output ontology entity relating to a clinical condition of the patient, wherein the ontology (i) comprises a set of ontology entities that represent clinical terms and (ii) specifies relationships between the clinical terms; and outputting data defining the one or more text spans and the respective output ontology entity for each of the one or more text spans.

The foregoing and other embodiments can each optionally include one or more of the following features, alone or in combination. The method may further include aggregating one or more clinical conditions related to one or more output ontology entities matched to one or more text spans in the one or more clinical notes; and generating a patient summary that organizes the aggregated clinical conditions. The unstructured text may include one or more of an abbreviation, a spelling error, language ambiguity, or hand-written text. The neural network may be a multi-task encoder-only transformer neural network. The multi-task encoder-only transformer neural network may be configured to perform a multi-class classification task. The multi-class classification task may be to classify one or more of a plurality of words in an input phrase into one of a plurality of categories including (i) problem, (ii) body part, (iii) qualifier, and (iv) procedure. The neural network may be a bidirectional Transformer encoder neural network.

For each of the one or more text spans, matching, using the text matcher, the text span with the respective ontology entity from the ontology may include: dividing the respective input phrase identified by the text span into a plurality of components; for each of the plurality of components, generating a set of component alternatives, wherein the component alternatives in the set (i) are ontology entities from the ontology and (ii) represent different ways to refer to a same concept that the component refers to, and wherein each component alternative in the set is associated with a component cost that represents a similarity in meaning between the component alternative and the component; generating, from the component alternatives generated for the plurality of components, a plurality of alternative writings for the respective input phrase identified by the text span, each alternative writing is associated with a phrase cost computed based on the costs of the component alternatives; and selecting, from the ontology, the respective output ontology entity that matches with an alternative writing that has a minimum phrase cost among the plurality of alternative writings.

Dividing the respective input phrase identified by the text span into the plurality of components may include dividing the respective input phrase into the plurality of components based on at least one of (i) one or more punctuation marks or (ii) one or more conjunctive words. For each of the plurality of components, generating a set of component alternatives may include: applying one or more transformations to the component, wherein the one or more transformations include at least one of (i) correcting the component, (ii) finding a synonym of the component, or (ii) stemming the component.

Generating, from the component alternatives generated for the plurality of components, the plurality of alternative writings for the respective input phrase identified by the text span may include: applying one or more transformations to the component alternatives generated for the plurality of components, wherein the one or more transformations include one or more of combining the component alternatives, adding a word to the component alternatives, dropping a word in the component alternatives, or changing an order of the component alternatives.

Aggregating one or more clinical conditions related to one or more output ontology entities matched to one or more text spans in the one or more clinical notes may include: grouping the one or more clinical conditions into one or more groups, wherein the one or more clinical conditions in each group are related to a same condition, removing, from the one or more groups, one or more clinical conditions that are ruled out, grouping the one or more groups into one or more sub-groups that each including clinical conditions related to each other; for each of the one or more sub-groups, classifying, using a classification neural network, the clinical conditions in the sub-group into a plurality of condition categories; and ranking the clinical conditions in each condition category according to a severity and recent of the clinical conditions. The plurality of condition categories may include one or more of active conditions, historical conditions, procedures, or symptoms. The text matcher may be a graph-based text matcher.

The subject matter described in this specification can be implemented in particular embodiments so as to realize one or more of the following advantages.

Clinical notes often contain useful information not documented in structured data. The unstructured nature of clinical notes can lead to critical patient-related information being missed. This is because existing methods for identifying clinical conditions from clinical notes often do not link identified entities relating to clinical conditions to an ontology and are therefore sensitive to abbreviations, spelling errors and language ambiguity. Another limitation of many existing solutions that use neural networks is that they are built on top of recurrent neural networks designed for solving name-entity recognition (NER) tasks, which often do not fully utilize nuanced linguistic signals. Thus, these neural networks may fail to perform tasks that demand a deep understanding of context of clinical notes in order to accurately identify clinical conditions. In addition, existing solutions operate at a note level and therefore are not able to aggregate clinical conditions in multiple clinical notes into a patient's overall summary that systematically organizes the patient's clinical conditions. These limitations decrease the utility of previous methods in the real-world.

To address the drawbacks of conventional techniques, the techniques described in this specification extract one or more text spans from the unstructured text of a clinical note using a neural network and match each text span with a respective output ontology entity in an ontology by using a text matcher. By maintaining an ontology that includes a large number of ontology entities that represent various clinical terms in different forms and specifies relationships between the clinical terms, and by matching each text span to a respective ontology entity, the techniques described herein allow more clinical conditions of the patient to be captured in a more accurate way and allow care providers to better understand their patients' conditions from clinical notes in comparison to previous techniques. In addition, the described techniques can be employed to build an end-to-end system that detects all clinical conditions on a clinical note and aggregates over the set of clinical conditions detected on all of the patient's clinical notes to produce a concise patient summary that organizes their clinical conditions according to one or more criteria (e.g., a severity and/or recent of the clinical conditions).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example multi-task neural network system.

FIG. 2 is a flow diagram of an example process for processing one or more clinical notes to generate, for each clinical note, data defining one or more text spans in the clinical note and the respective output ontology entity for each text span.

FIG. 3 is a flow diagram of an example process for matching each text span to a respective ontology entity from the ontology.

FIG. 4 illustrates an example graph that is used to find the closest ontology entity in the ontology to an input string.

Like reference numbers and designations in the various drawings indicate like elements.

### DETAILED DESCRIPTION

This specification describes a multi-task neural network system implemented as computer programs on one or more computers in one or more locations for matching unstructured text in clinical notes to ontology entities in an ontology in order to determine clinical conditions of a patient.

The ontology includes a set of ontology entities that represent clinical terms and specifies relationships between the clinical terms. The ontology captures the clinical knowledge required for the system to detect clinical conditions that are implied and/or mentioned in the clinical notes. For example, the ontology includes an ontology entity that represents an abbreviation of a clinical condition (e.g., “IDDM” or “iddm”), another ontology entity that explains the abbreviation (e.g., “insulin dependent diabetes mellitus”) and another ontology entity that represents an acronym of the explanation (e.g., diabetes mellitus type 1). As another example, the ontology includes ontology entities that represent a short form and a long form of a clinical term that refer to the same clinical condition (e.g., “Miller” may refer to “Miller Fisher syndrome”). The ontology may also capture knowledge about related clinical conditions (e.g., “biventricular congestive heart failure” is related to “right heart failure”) and about possible complications of certain conditions (e.g., “diabetic nephropathy” is a complication of “diabetes mellitus”). Example ontologies include SNOMED-CT (described in Kevin Donnelly et al. 2006. Snomed-ct: The advanced terminology and coding system for ehealth. Studies in health technology and informatics, 121:279.), MeSH (described in Carolyn E Lipscomb. 2000. Medical subject headings (mesh). Bulletin of the Medical Library Association, 88(3):265.), ICD-10-CM (described in World Health Organization. 2015. International classification of diseases, tenth revision, (icd-10).), and UMLS (described in Donald A B Lindberg, Betsy L Humphreys, and Alexa T McCray. 1993. The unified medical language system. Yearbook of medical informatics, 2(01):41-51.).

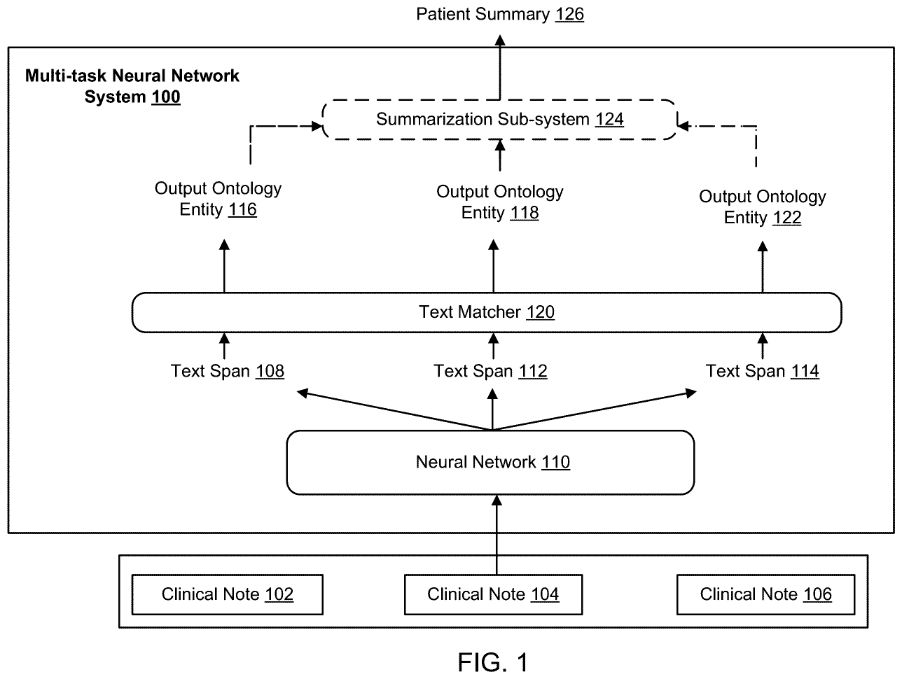

FIG. 1 shows an example multi-task neural network system 100. The system 100 is an example of a system implemented as computer programs on one or more computers in one or more locations, in which the systems, components, and techniques described below can be implemented.

The system 100 is configured to receive one or more clinical notes (e.g., clinical notes 102, 104, and 106) associated with a patient. Each of the one or more clinical notes includes unstructured text.

The unstructured text in the clinical note may include narrative descriptions or written records of a patient's medical history, symptoms, examinations, diagnoses, treatments, and/or other medical-related information. The unstructured text may be entered by healthcare providers, such as physicians, nurses, or other medical professionals, into the patient's medical record. Unlike structured data, which is organized and follows a predefined format or template, unstructured clinical notes are free-text in nature, allowing healthcare providers to document a patient's condition and treatment in a more timely, flexible, and detailed manner.

The unstructured text may include one or more of an abbreviation, a spelling error, language ambiguity, or hand-written text.

The multi-task neural network system 100 includes a neural network 110, a text matcher 120, and a summarization sub-system 124. The text matcher 120 is a software-based system, subsystem, or process that is programmed to perform one or more specific functions. Generally, the text matcher 120 is implemented as one or more software modules or components, installed on one or more computers in one or more locations. Similarly, the summarization sub-system 124 is a software-based system, subsystem, or process that is programmed to perform one or more specific functions. Generally, the summarization sub-system 124 is implemented as one or more software modules or components, installed on one or more computers in one or more locations.

For each of the clinical notes, the neural network 110 is configured to extract one or more text spans from the unstructured text of the clinical note. For example, as shown in FIG. 1, the neural network 110 is configured to extract text spans 108, 112, and 114 from the unstructured text of the clinical note 104. Each of the one or more text spans identifies a respective input phrase in the unstructured text.

In some implementations, the neural network 110 is a multi-task encoder-only transformer neural network. For example, the neural network can be a Bidirectional Encoder Representations from Transformers (BERT) neural network. In these implementations, the multi-task encoder-only transformer neural network is configured to perform a multi-class classification task. The multi-class classification task is to classify a word or a phrase that includes a plurality of consecutive words into one of a plurality of categories. The plurality of categories may include, but not be limited to, (i) Problem, (ii) Body Part, (iii) Qualifier, (iv) Procedure, and (v) Unknown.

The multi-task encoder-only transformer neural network has been trained to classify a word or a group of consecutive words into one of a plurality of categories.

Given the unstructured text, the multi-task encoder-only transformer neural network is configured to generate all possible candidate text spans. Each candidate text span is a word or a group of consecutive words in the unstructured text.

For each candidate text span, the trained multi-task encoder-only transformer neural network is configured to generate a probability distribution over the plurality of categories. The probability includes, for each category, a respective probability indicating the likelihood that the candidate text span belongs to the category. The system 100 selects the category with the highest probability to be the category of the candidate text span. The candidate text span can be assigned to a valid category if the candidate text span belongs to the Problem, Body Part, Qualifier, or Procedure (not the Unknown category).

The system 100 outputs all candidate text spans with valid categories as the extracted text spans from the unstructured text.

For instance, considering unstructured, hand-writing text that states “urgent surgery for diabetic foot ulcer.” The multi-task encoder-only transformer neural network 110 is configured to extract multiple text spans, each corresponding to a respective input phrase from the unstructured text: “urgent,” “surgery,” “diabetic,” “foot,” and “ulcer.” The multi-task encoder-only transformer neural network 110 classifies each word in the unstructured text into one of the plurality of categories and label the word with the corresponding category. For example, “urgent” and “diabetic” are labelled as Qualifier, “foot” is labelled as Body Part, “ulcer” is labelled as Problem, and “surgery” is labelled as Procedure.

In some implementations, the multi-task encoder-only transformer neural network 110 is configured to perform an existence determination task to generate, for each label assigned to each word, an existence signal that indicates whether the label is Present or Absent. For example, in “ruled out cancer,” the word “cancer” is labeled as Problem but with an existence signal indicating that the label Problem is Absent because the cancer has been ruled out. As another example, if a word(s) is labeled as Problem but the problem is a known side effect of treatments of the patient, the neural network 110 may determine that the word labeled as Problem has an existence signal indicting that the label is Absent. The existence signals generated by the neural network 110 are used later by the summarization sub-system 124 to group clinical conditions before adding them to a patient summary 126.

In some implementation, the multi-task encoder-only transformer neural network 110 is configured to perform a relation determination task that determines a relationship between different text spans. For example, the neural network 110 is configured to determine a position of a word(s) labelled as Body Part and/or Qualifier relative to a position of a word(s) labelled as Problem and/or Procedure. For example, in an input phrase that includes “diabetic foot ulcer,” the neural network 110 determines that “ulcer” will have a LEFT_HAND_SIDE label, because “diabetic foot” is on the left hand side of “ulcer.” This relational information is later used to map an annotated term to the most accurate ontology entity.

For example, consider the following input text: “Patient was admitted to the ER with a broken ulna on their left arm.” The phrase “left arm” is a modifier of “broken ulna.” The neural network 110 determines relational information indicating that the two disjoint spans of text (“left arm” and “broken ulna”) are related. The system 100 may consider them as one entry when finding a match in the ontology. For example, the system may determine the following sequence of alternatives of “left arm” and “broken ulna:”
⇒left arm+ulna broken
⇒left arm+ulnar broken
⇒left arm+ulnar fracture
⇒left_+ulnar fracture

“Left” and “ulnar fracture” is the most accurate ontology entity that matches the input text. Without the relational information, the system 100 only matches “broken ulna” to an ontology entity. In this case, the result would be “ulnar fracture” that is less specific than “left ulnar fracture.”

After the text spans have been extracted from the unstructured text, the text matcher 120 is configured to match each of the one or more text spans to a respective output ontology entity from an ontology. The respective output ontology entity relates to a clinical condition of the patient. For example, as shown in FIG. 1, the text matcher 120 matches the text span 108 to an output ontology entity 116, matches the text span 112 to an output ontology entity 118, and matches the text span 114 to an output ontology entity 122.

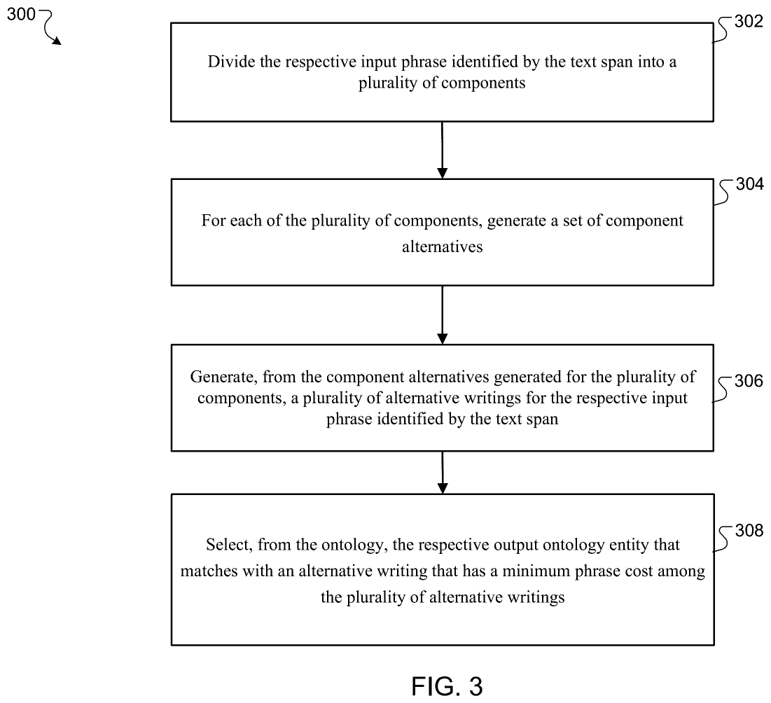

The process for matching a text span to a respective ontology entity from the ontology is described in more detail below with reference to FIG. 3.

After each text span is matched to a respective output ontology entity which relates to a clinical condition of the patient, the summarization model 124 aggregates all of the clinical conditions related to the output ontology entities and presents them in a patient summary 126. In particular, to aggregate the clinical conditions, the summarization sub-system 124 groups the clinical conditions into one or more groups, in which clinical conditions in the same group are related to the same condition. Further, the summarization sub-system 124 removes, from the one or more groups, one or more clinical conditions that are ruled out by using the existence signals generated by the multi-task encoder-only transformer neural network 110 as described above. For example, the summarization sub-system 124 may drop a condition because the condition was ruled out, or drop a condition that the patient never had because the condition is mentioned in the clinical note as a known side effect of treatments of the patient.

In some implementations, the summarization sub-system 124 is configured to group the one or more groups into one or more sub-groups that each include clinical conditions related to each other. More specifically, a sub-group is a collection of clinical conditions composed of one or more anchor ontology entities and their corresponding descendants in the ontology. For example, if a patient's medical notes mention Systolic Heart Failure, Diastolic Heart Failure, Acute Heart Failure, and Acute Diastolic Heart Failure, the summarization model 124 groups those mentions under a Heart Failure sub-group, even if “Heart Failure” itself was not explicitly mentioned in the patient's record. In this example, Heart Failure is an anchor ontology entity, used to group together more specific clinical conditions as defined by is—a relation of the same ontology that includes the Heart Failure ontology entity.

For each of the one or more sub-groups, the summarization sub-system 124 is configured to classify, using a classification neural network, the clinical conditions in the sub-group into a plurality of condition categories. The plurality of condition categories may include one or more of active conditions, historical conditions, procedures, or symptoms.

In addition, the summarization sub-system 124 is configured to rank the clinical conditions in each condition category according to a severity and recency of the clinical conditions. The summarization sub-system 124 may determine a level of severity of each clinical condition based on at least one of (i) one or more signals related to the clinical condition from the ontology, (ii) one or more signals from the surrounding text such as existence signals and relational information, or (iii) one or more manually curated expert annotations of the clinical condition. The summarization sub-system 124 may determine the recency of each clinical condition based on a date on which the clinical condition was first mentioned in a clinical note.

Based on the grouping, categorizing, and ranking results, the summarization sub-system 124 organizes the clinical conditions into appropriate sub-group and categories and displays the organized clinical conditions in the patient summary 126. The sub-system 124 displays the clinical conditions according to their rankings, i.e., the more severe and more recent clinical conditions are displayed in higher positions in the patient summary to get more attention from healthcare providers who read the patient summary 126.

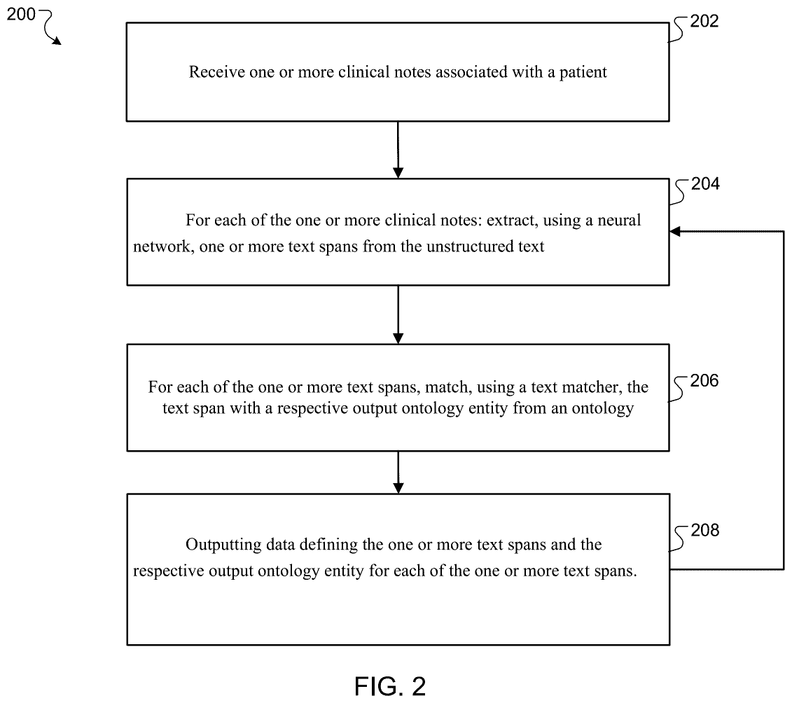

FIG. 2 is a flow diagram of an example process 200 for processing one or more clinical notes to generate, for each clinical note, data defining one or more text spans in the clinical note and the respective output ontology entity for each text span. For convenience, the process 200 will be described as being performed by a system of one or more computers located in one or more locations. For example, a neural network system, e.g., the multi-task neural network system 100 of FIG. 1, appropriately programmed in accordance with this specification, can perform the process 200.

The system receives one or more clinical notes associated with a patient (step 202). Each of the one or more clinical notes includes unstructured text. The unstructured text may include one or more of an abbreviation, a spelling error, language ambiguity, or hand-written text.

For each of the one or more clinical notes, the system performs steps 204-208 as follows.

The system extracts, using a neural network, one or more text spans from the unstructured text of the clinical note (step 204). Each of the one or more text spans identifies a respective input phrase in the unstructured text.

In some implementations, the neural network is a multi-task encoder-only transformer neural network. For example, the neural network is a bidirectional Transformer encoder neural network.

For each of the one or more text spans, the system matches, using a text matcher, the text span with a respective output ontology entity from an ontology (step 206). The respective output ontology entity relates to a clinical condition of the patient. In some implementations, the text matcher is a graph-based text matcher.

To match the text span with the respective ontology entity from the ontology, the system may divide, using the text matcher, the respective input phrase identified by the text span into a plurality of components. For each of the plurality of components, the system may generate, using the text matcher, a set of component alternatives. The system may generate, from the component alternatives generated for the plurality of components, a plurality of alternative writings for the respective input phrase identified by the text span. Each alternative writing is associated with a phrase cost computed based on the costs of the component alternatives. The system may select, from the ontology, the respective output ontology entity that matches with an alternative writing that has a minimum phrase cost among the plurality of alternative writings.

The process for matching a text span to a respective ontology entity from the ontology is described in more detail below with reference to FIG. 3. The system outputs data defining the one or more text spans and the respective output ontology entity for each of the one or more text spans (step 208).

In some implementations, the system aggregates one or more clinical conditions related to one or more output ontology entities matched to one or more text spans in the one or more clinical notes, and generates a patient summary that organizes the aggregated clinical conditions.

In some implementations, to aggregate the one or more clinical conditions, the system groups the one or more clinical conditions into one or more groups, in which the one or more clinical conditions in each group are related to a same condition. The system removes, from the one or more groups, one or more clinical conditions that are ruled out. The system groups the one or more groups into one or more sub-groups that each including clinical conditions related to each other.

In some implementations, for each of the one or more sub-groups, the system classifies, using a classification neural network, the clinical conditions in the sub-group into a plurality of condition categories. The plurality of condition categories may include one or more of active conditions, historical conditions, procedures, or symptoms.

In addition, in some implementations, the system ranks the clinical conditions in each condition category according to a severity and recentness of the clinical conditions.

Based on the grouping, categorizing, and ranking results, the system can organize the clinical conditions into appropriate sub-groups and categories and display the organized clinical conditions in the patient summary. The system can also display the clinical conditions according to their rankings, i.e., the more severe and more recent clinical conditions are displayed in higher positions in the patient summary to get more attention from healthcare providers who read the patient summary.

FIG. 3 is a flow diagram of an example process 300 for matching each text span to a respective ontology entity from the ontology. For convenience, the process 300 will be described as being performed by a system of one or more computers located in one or more locations. For example, a neural network system, e.g., the multi-task neural network system 100 of FIG. 1, appropriately programmed in accordance with this specification, can perform the process 300.

The system divides the respective input phrase identified by the text span into a plurality of components of different types(step 302). The different types may include one or more of: a comma, a connector (e.g., “due to,” “because,” or “of”), a negating connector (e.g., “not” or “without”), a phrase (e.g., “acute” or “bilateral”), or a qualifier (e.g., “chronic” or “diabetic”).

For each of the plurality of components that include phrases, the system generates a set of component alternatives using a text matcher (step 304).

For a given component, the component alternatives in the set (i) are ontology entities from the ontology and (ii) represent different ways to refer to the same concept that the component refers to.

To generate a set of component alternatives for a given component, the system applies, using the text matcher, one or more transformations to the component.

In particular, in some implementations, the system drops an optional word in the component to generate a component alternative.

In some implementations, the system uses one or more synonyms of the component as component alternatives. The system may determine one or more synonyms of a component by detecting differences in aliases that refer to a same condition. For example, “Chronic kidney disease” and “Chronic renal disease” refer to the same condition in the ontology. The system detects that the term “kidney” is different from “renal” in these two aliases. Thus, the system determines that “kidney” is a candidate component alternative of the component “renal.” In some implementations, the system takes into account common replacements which are frequently used in medical notes but are not part of the ontology. For example, “IDDM” is a component alternative of “Insulin-dependent Diabetes Mellitus.”

In some implementations, the system generates component alternatives from a stem of the component (e.g., generating the component alternative “diabetic” from the stem “diabet” in the component “diabetes”).

For example, for a component called “malignant tumor,” the text matcher generates “cancer” as a component alternative. Similarly, the text matcher can generate “pulmonary” as a component alternative of “lung,” “renal” as a component alternative of “kidney,” and “diabetes” as a component alternative of “diabetes mellitus.”

Each component alternative in the set is assigned a component cost that represents a similarity in meaning between the component alternative and the component. For example, replacing “ii” with “2” has a lower cost compared to replacing “infectious disease” with “infection.”

The system generates, from the component alternatives generated for the plurality of components, using the text matcher, a list of alternative writings for the respective input phrase identified by the text span (step 306). Each alternative writing is associated with a phrase cost computed based on the costs of the component alternatives.

The alternative writing may include the original component as it appears in the input phrase and alternative wordings that are based on, for example, one or more of synonyms, dropping optional phrases, or stemming. For example, “diabetes” is a synonym of “diabetes mellitus,” “diabet” is the canonical form of “diabetes.”

To generate the list of alternative writings, the system applies, using the text matcher, one or more transformations to the component alternatives generated for the plurality of components. The one or more transformations include one or more of combining the component alternatives, adding a word to the component alternatives, dropping a word in the component alternatives, or changing an order of the component alternatives.

The component alternatives include different combinations of the options created for the components previously generated. In some implementations, the text matcher can perform phrase level transformations with additional cost. For example, the connector “due to” allows a transformation of dropping itself and the possibility of swapping both of its sides. For instance, the text matcher may generate for “coma due to diabetes mellitus” component alternatives such as “coma diabetes mellitus” and “diabetes mellitus coma” (each with a component cost). The final (phrase level) cost is set to be the costs sum of all replacements and operations applied to the input string.

The system selects, from the ontology, the respective output ontology entity that matches with an alternative writing that has a minimum phrase cost among the plurality of alternative writings (step 308).

In particular, the ontology is represented as a graph with nodes representing ontology entities and edges representing a relationships between ontology entities. Each edge is associated with a component cost. When one or more component alternatives are generated for a particular component, new nodes representing the one or more component alternatives are added to the graph. One or more new edges between the one or more component alternatives and the particular component, and between the one or more component alternatives themselves are added to the graph. Each of the new edges also has a component cost. A component cost associated with an edge may be computed by applying a distance function (e.g., Euclidean distance or Hamming distance) to both nodes that the edge connects together. The system computes the phrase cost for each alternative writing by summing the component costs associated with edges along a path that connects the original component with the alternative writing. The alternative writing that has the minimum phrase cost among the plurality of alternative writings is the respective output ontology matched to the text span.

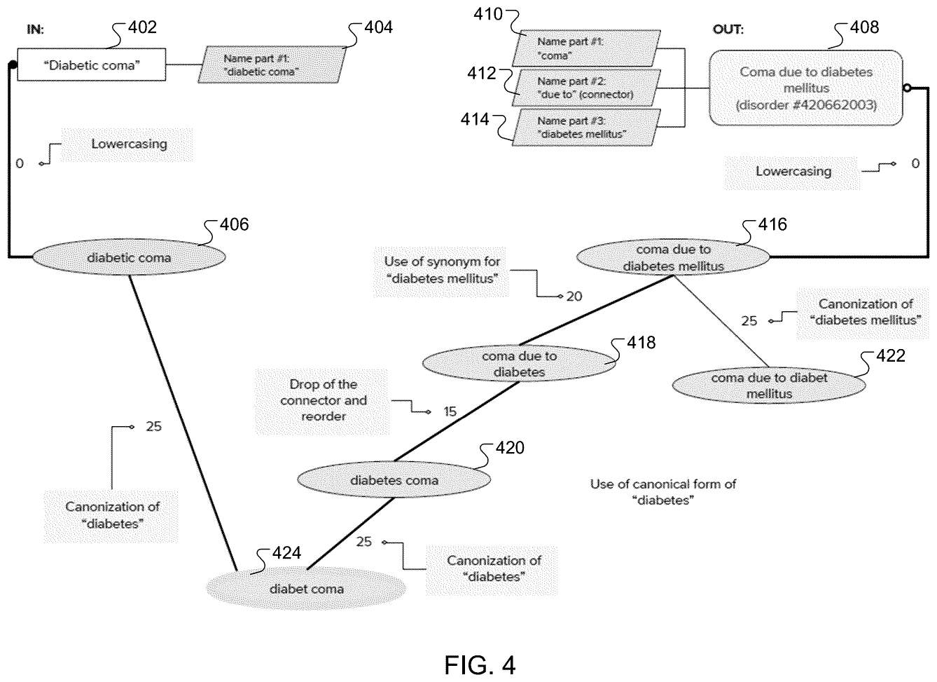

FIG. 4 illustrates an example graph that is used to find the closest ontology entity in the ontology to an input string.

As shown in FIG. 4, an input string “Diabetic coma” (402) is divided into a single component named “diabetic coma” (404). The ontology entity “Coma due to diabetes mellitus” is divided into components “Coma” (410), “diabetes mellitus” (412) and the connector “due to” (414). The system generates component alternatives of each component both for the input string and the ontology entity or entities, given in ovals 406, 416, 418, 420, 424, and 422 accordingly. The system finds the shortest path between the input string 402 and the ontology, that is “Coma due to diabetes mellitus” (408) with the minimum phrase cost of 85. The system matches the input string “Diabetic coma” (402) to the ontology entity “Coma due to diabetes mellitus” (408).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method comprising:
receiving one or more clinical notes associated with a patient, wherein each of the one or more clinical notes includes unstructured text;
for each of the one or more clinical notes:
extracting, using a neural network, one or more text spans from the unstructured text, each of the one or more text spans identifying a respective input phrase in the unstructured text;
for each of the one or more text spans, matching, using a text matcher, the text span with a respective output ontology entity from an ontology, the respective output ontology entity relating to a clinical condition of the patient, wherein the ontology (i) comprises a set of ontology entities that represent clinical terms and (ii) specifies relationships between the clinical terms; and
outputting data defining the one or more text spans and the respective output ontology entity for each of the one or more text spans.

2. The method of claim 1, further comprises:
aggregating one or more clinical conditions related to one or more output ontology entities matched to one or more text spans in the one or more clinical notes; and
generating a patient summary that organizes the aggregated clinical conditions.

3. The method of claim 1, wherein the unstructured text includes one or more of an abbreviation, a spelling error, language ambiguity, or hand-written text.

4. The method of claim 1, wherein the neural network is a multi-task encoder-only transformer neural network.

5. The method of claim 4, wherein the multi-task encoder-only transformer neural network is configured to perform a multi-class classification task.

6. The method of claim 5, wherein the multi-class classification task is to classify one or more words in an input phrase into one of a plurality of categories including (i) problem, (ii) body part, (iii) qualifier, and (iv) procedure.

7. The method of claim 4, wherein the multi-task encoder-only transformer neural network is a Bidirectional Encoder Representations from Transformers (BERT) neural network.

8. The method of claim 1, wherein, for each of the one or more text spans, matching, using the text matcher, the text span with the respective ontology entity from the ontology comprises:
dividing the respective input phrase identified by the text span into a plurality of components;
for each of the plurality of components, generating a set of component alternatives, wherein the component alternatives in the set (i) are ontology entities from the ontology and (ii) represent different ways to refer to a same concept that the component refers to, and wherein each component alternative in the set is associated with a component cost that represents a similarity in meaning between the component alternative and the component;
generating, from the component alternatives generated for the plurality of components, a plurality of alternative writings for the respective input phrase identified by the text span, each alternative writing is associated with a phrase cost computed based on the costs of the component alternatives; and
selecting, from the ontology, the respective output ontology entity that matches with an alternative writing that has a minimum phrase cost among the plurality of alternative writings.

9. The method of claim 8, wherein for each of the plurality of components, generating a set of component alternatives comprises: applying one or more transformations to the component, wherein the one or more transformations include at least one of (i) correcting the component, (ii) finding a synonym of the component, or (ii) stemming the component.

10. The method of claim 8, wherein generating, from the component alternatives generated for the plurality of components, the plurality of alternative writings for the respective input phrase identified by the text span comprises:
applying one or more transformations to the component alternatives generated for the plurality of components, wherein the one or more transformations include one or more of combining the component alternatives, adding a word to the component alternatives, dropping a word in the component alternatives, or changing an order of the component alternatives.

11. The method of claim 2, wherein aggregating one or more clinical conditions related to one or more output ontology entities matched to one or more text spans in the one or more clinical notes comprises:
grouping the one or more clinical conditions into one or more groups, wherein the one or more clinical conditions in each group are related to a same condition,
removing, from the one or more groups, one or more clinical conditions that are ruled out,
grouping the one or more groups into one or more sub-groups that each including clinical conditions related to each other;
for each of the one or more sub-groups, classifying, using a classification neural network, the clinical conditions in the sub-group into a plurality of condition categories; and
ranking the clinical conditions in each condition category according to a severity and recent of the clinical conditions.

12. The method of claim 11, wherein the plurality of condition categories includes one or more of active conditions, historical conditions, procedures, or symptoms.

13. The method of claim 1, wherein the text matcher is a graph-based text matcher.

14. A system comprising one or more computers and one or more storage devices storing instructions that, when executed by the one or more computers, cause the one or more computers to perform the operations comprising:
receiving one or more clinical notes associated with a patient, wherein each of the one or more clinical notes includes unstructured text;
for each of the one or more clinical notes:
extracting, using a neural network, one or more text spans from the unstructured text, each of the one or more text spans identifying a respective input phrase in the unstructured text;
for each of the one or more text spans, matching, using a text matcher, the text span with a respective output ontology entity from an ontology, the respective output ontology entity relating to a clinical condition of the patient, wherein the ontology (i) comprises a set of ontology entities that represent clinical terms and (ii) specifies relationships between the clinical terms; and
outputting data defining the one or more text spans and the respective output ontology entity for each of the one or more text spans.

15. The system of claim 14, wherein the operations further comprise:
aggregating one or more clinical conditions related to one or more output ontology entities matched to one or more text spans in the one or more clinical notes; and
generating a patient summary that organizes the aggregated clinical conditions.

16. The system of claim 14, wherein, for each of the one or more text spans, the operations for matching, using the text matcher, the text span with the respective ontology entity from the ontology comprise:
dividing the respective input phrase identified by the text span into a plurality of components;
for each of the plurality of components, generating a set of component alternatives, wherein the component alternatives in the set (i) are ontology entities from the ontology and (ii) represent different ways to refer to a same concept that the component refers to, and wherein each component alternative in the set is associated with a component cost that represents a similarity in meaning between the component alternative and the component;
generating, from the component alternatives generated for the plurality of components, a plurality of alternative writings for the respective input phrase identified by the text span, each alternative writing is associated with a phrase cost computed based on the costs of the component alternatives; and
selecting, from the ontology, the respective output ontology entity that matches with an alternative writing that has a minimum phrase cost among the plurality of alternative writings.

17. The system of claim 16, wherein for each of the plurality of components, generating a set of component alternatives comprises: applying one or more transformations to the component, wherein the one or more transformations include at least one of (i) correcting the component, (ii) finding a synonym of the component, or (ii) stemming the component.

18. One or more non-transitory computer storage media storing instructions that, when executed by one or more computers, cause the one or more computers to perform the operations comprising:
receiving one or more clinical notes associated with a patient, wherein each of the one or more clinical notes includes unstructured text;
for each of the one or more clinical notes:
extracting, using a neural network, one or more text spans from the unstructured text, each of the one or more text spans identifying a respective input phrase in the unstructured text;
for each of the one or more text spans, matching, using a text matcher, the text span with a respective output ontology entity from an ontology, the respective output ontology entity relating to a clinical condition of the patient, wherein the ontology (i) comprises a set of ontology entities that represent clinical terms and (ii) specifies relationships between the clinical terms; and
outputting data defining the one or more text spans and the respective output ontology entity for each of the one or more text spans.

19. The one or more non-transitory computer storage media of claim 18, wherein the operations further comprise:
aggregating one or more clinical conditions related to one or more output ontology entities matched to one or more text spans in the one or more clinical notes; and
generating a patient summary that organizes the aggregated clinical conditions.

20. The one or more non-transitory computer storage media of claim 18, wherein, for each of the one or more text spans, the operations for matching, using the text matcher, the text span with the respective ontology entity from the ontology comprise:
dividing the respective input phrase identified by the text span into a plurality of components;
for each of the plurality of components, generating a set of component alternatives, wherein the component alternatives in the set (i) are ontology entities from the ontology and (ii) represent different ways to refer to a same concept that the component refers to, and wherein each component alternative in the set is associated with a component cost that represents a similarity in meaning between the component alternative and the component;
generating, from the component alternatives generated for the plurality of components, a plurality of alternative writings for the respective input phrase identified by the text span, each alternative writing is associated with a phrase cost computed based on the costs of the component alternatives; and
selecting, from the ontology, the respective output ontology entity that matches with an alternative writing that has a minimum phrase cost among the plurality of alternative writings.
